(19) United States
(12) Patent Application Publication
(10) Pub. No.: US 2026/0166195 A1
(43) Pub. Date: Jun. 18, 2026

(54) Mondar Pain Barrier Non-Pharmaceutical Material for Chronic Joint Pains

(71) Applicant: Darlene R. Bembry-Miles, Boardman, OH (US)

(72) Inventor: Darlene R. Bembry-Miles, Boardman, OH (US)

(21) Appl. No.: 19/368,684

(22) Filed: Oct. 24, 2025

(51) Int. Cl. A61L 15/24; A61F 13/00

(52) U.S. Cl. CPC A61L 15/24; A61F 13/00063

(57) ABSTRACT

a. This application is being submitted to the U.S. Patent and Trademark Office for review, processing, and approval of the issuance of a Utility Nonprovisional Patent and Trademark of a ‘newly discovered non-obvious use’ of an existing material that can be utilized to alleviate chronic joint pains. This newly discovered non-pharmaceutical material, does not require FDA approval. When applied to the skin area of a human's painful joints the material has alleviated joint pain, helped to stabilize the joint, reduced swelling around the joint, and helped to promote joint healing. This discovery can help millions who suffer with chronic joint pains return to a productive life. This non-medicated, non-pharmaceutical commercial material when applied to the skin area of a painful joint has been used and tested successfully for two (2) to alleviate joint pain, as a chronic pain-management process. The material and ‘application process’ of the material has completely stop joint pain, and reduced swelling while helping to promote the joint healing process without medication in the human body.

### BACKGROUND

b. In researching this existing material it was found that the material was originally patented in in 1925, two German businessmen Schmidt and Dykerhoff filed for patents on reflective surfaces for use as building insulation because recent improvements in technology allowed low emissivity aluminum foil to be commercially viable. This became the launching pad for radiant barrier and reflective insulation around the world, and within the next 15 years, millions of square feet of radiant barrier were installed in the US alone. This material has not been utilized as a pain-management ‘Application-Process’ until my discovery and non-obvious ‘new use’ of this material.

According to 2024 data from the CDC, nearly 1 in 4 adults in the United States—24.3% of the population—live and suffer with chronic pain. A staggering 8.5% of adult Americans experience high-impact chronic pain, significantly impairing their ability to work, care for loved ones, or engage in daily activities. As human beings our ability to age gracefully and productively is being removed by these disabling chronic pains.

My goal is to introduce my discovery of this non-pharmaceutical material and the successful ‘application process’ and procedure for this ‘new use’ of my approved process of use, of this building material as an effective way to manage pain and reduce chronic joint pains.

Despite its vast prevalence, chronic pain remains one of the most overlooked and underfunded public health crises of our time. For those who endure it, chronic pain is not just a symptom but a complex and debilitating disease in itself that demands more research, better treatments, and systemic understanding (U.S. Pain Foundation 2025). I have now discovered an existing commercial material that will help human beings to manage their chronic joint pains without medication or the deterioration of the bodies joints that is leading to millions of human joints being replaced annually.

With my over thirty years in information systems technology, with the last fifteen years of consulting in healthcare technology systems I, (the inventor and discoverer) of this non-medicated material and ‘application process’ now used for pain-management, I have realized the harmful impact of the ‘foreign metals’ being released into our air, on our skin, and in our bodies. Many healthcare facilities that I have worked with has moved to the surgical extraction and removal of these many metals from our joints as a treatment option yet it does not mitigate the human exposure to the metals. My discovered new use and ‘application process’ can help to protect healthy joints and allow many to remain productive in their lives.

The metals referenced are some of the metals that we consume while others are sometimes-airborne materials that negatively affect our bodies in other ways as well. As those metals and foreign micro metal objects settle under our skin or into our joints, the damage to our bodies chronically begins. The joint pains, in most, occur when those metals and objects penetrate our skin and begin to shift within our joints.

These chronic joint pains are increasingly occurring in millions of Americans. Human beings who are now suffering with increased joint pains and ultimately joint damage that is requiring in over 25 million Americans and now resulting in debilitating high level of joint-replacements.

My product discovery has help to alleviate and stop the joint pains, and is now being used as a ‘Pain Barrier’ over healthy joints for protection. The discovered material has helped to stop the shifting of metals in the joints and immediately decreased pain. By utilizing this material as a ‘Pain Barrier’ the joint pain ended and no further joint deterioration occurred.

The increased creation of nano technology, nano-sensors that consist of metals and biological chemicals as just two of the many developed components in the U.S. These nano-sensor micro metals are a source of the many metals that negatively affect our bodies. These nano-sensor technologies are being released all around our bodies every day, in our air, our waters and over our agriculture (farms and foods).

Nano-technologies, ‘nano sensors’ are being created smaller and smaller and are now 80,000 to 100,000 times smaller than a strain of hair and making great impact in our skies as written in one of the ‘sciencenewstoday.org’ publication referencing Mar. 26, 2025 article (amongst many other articles and publications) written https://www.sciencenewstoday.org/nanotechnology-small-tech-big-impact, and as documented in a host of many other publications as well.

### Methods

c. The method of ‘new’ use of this product as a ‘Pain Barrier’ is to place the material over the skin of the ‘pain area or joint’ location to experience immediate pain relief and pain-management without medication.

The testing was conducted over two years by placing the product ‘application process’ covering the front and back of the knee joint. Additional use and testing were to place the material in the ‘application process’ over the front and back of the shoulder area to treat Fibromyalgia pain. Additional application was to apply a certain amount of the material over the lower back to treat Sciatic nerve pain.

The material has also been used on the shoulder rotator-cuff in the same manner. The material ‘application process’ has also been used for spine pain by placing the material on the center of the spine and back to alleviate pain. All resulting in immediate pain relief.

### Findings

This significant discovery found that there was a fragment of an unknown micro metal fragment in the knee that the application process now entitled ‘Mondar Pain Barrier’ material stabilize, as well as immediately alleviated pain, reduce swelling in and around the knee area, and promoted the healing process.

The ‘Mondar Pain Barrier’ ‘application process’ acts as a shield which protects the joint from additional damage, while helping to stabilize the joint and promoting the healing process. In addition, the Pain Barrier stopped the exposure of the joint to additional exterior air-borne micro metals while acting as a shield over the joint and skin.

The new use of this commercial material is reducing exposure to additional environmental, or external metals materials such as ‘metal nano sensor’ technology micro particles. The nano sensor technology that is released into our U.S. air that penetrates the human body which is also used to monitor U.S. Agriculture (farms/growing foods). This same micro-metal nano sensor technology that is being utilized by many researchers who are also utilizing micro nano metals/technology in their testing in our air and in various forms.

Nano sensor technology particles that are being designed at an alarming rate and are now 80,000 to 100,000 times smaller than a strain of hair. The penetration of these metal materials into an already painful human joint, or new joint (pain) resulting in chronic pain in humans today.

### Discussion

This non-pharmaceutical material and my ‘application process’ does not required FDA approval of the use of the Double-sided Foil Radiant Barrier insulation material as a ‘Pain Barrier’ in application for pain-management for the human body and skin. I have discovered this new and non-oblivious successful use of this material that alleviates and stops pain while reducing swelling in joints and promotes the healing process in human body joints.

The new and successful use of this discovered building material can help many to control chronic pains by blocking movement of metals within the joints and body, and allow human beings to become pain-free and more productive. The use of the material as a pain-management material will not require a prescription and will lower medical cost while allowing people to age gracefully, and save money on medications while remaining productive in their lives.

### SPECIFICATIONS

a. Durable and puncture-resistant, this insulation requires no maintenance and includes advanced RFID blocking technology for added protection.
b. At ¼ inch thick-two layers of polyethylene industrialized air bubbles bonded between two layers of highly reflective metalized aluminum polyester film. Industrialized strength, lightweight yet durable material designed to hold in place, without tearing.
c. Does not compress, collapse or disintegrate. Reflects 97% of radiant heat, emits less than 3% of heat, Class 1/Class A fire rated radiant barrier that has passed the ASTM E84 test & ASTM E2599.
d. Non-toxic, non-carcinogenic, fiber-free, safe to handle and does not require protective clothing or respirators use or to apply the material.
e. Easy to apply and maintenance free, cuts easily with utility knife or scissors.
f. No itchy fibers.
g. Not affected by moisture or humidity. Inhibits condensation and does not promote the growth of fungi, mold or mildew.
h. Made in USA/Ships from USA.
i. Two layers of polyethylene industrialized air bubbles bonded between two layers of highly reflective metalized aluminum polyester film. Industrialized strength, lightweight yet durable material designed to hold without tearing.
j. Does not compress, collapse or disintegrate. Reflects 97% of radiant heat, emits less than 3% of heat, Class 1/Class A fire rated radiant barrier that has passed the ASTM E84 test & ASTM E2599.
k. Non-toxic, non-carcinogenic, fiber-free, safe to handle and does not require protective clothing or respirators to utilize.
l. Easy to apply, cuts easily with utility knife or scissors and applies to the human body no itchy fibers.
m. Not affected by moisture or humidity. Inhibits condensation and does not promote the growth of fungi, mold or mildew.

### BRIEF DESCRIPTION OF APPLICATION

A material comprising:
a. An exterior molded cut designed as an outer shell comprised of one to two sheets of the radiant barrier to the body joint;
b. A method of directly applying the pain barrier to the outer skin-body joint pain area in front and back of the body joint;
c. Inserting said material between under garments to secure the application of material;
d. Material can be cleansed with water and placed back on the body.

1. A material comprising:
a. An exterior molded cut designed as an outer shell of comprised of one to two sheets of the radiant barrier to the body;
b. A method of directly applying the barrier to the outer body joint pain area in front and back of the body joint;
c. Applying said material to painful joint areas, over the skin and held between under garments to secure the application of material alleviates pain;
d. Material can be cleansed with water and placed back on the body.
